(19) United States
(12) Patent Application Publication
(10) Pub. No.: US 2019/0008423 A1
(43) Pub. Date: Jan. 10, 2019

(54) LIVING BODY OBSERVATION SYSTEM

(71) Applicant: OLYMPUS CORPORATION, Tokyo (JP)

(72) Inventor: Kei KUBO, Tokyo (JP)

(73) Assignee: OLYMPUS CORPORATION, Tokyo (JP)

(21) Appl. No.: 16/131,161

(22) Filed: Sep. 14, 2018

Related U.S. Application Data
(63) Continuation of application No. PCT/JP2017/008107, filed on Mar. 1, 2017.

(30) Foreign Application Priority Data
May 19, 2016 (JP) 2016-100593

(51) Int. Cl. A61B 5/1459; A61B 1/05; A61B 1/00; A61B 1/06

(52) U.S. Cl. CPC A61B 5/1459; A61B 1/0638; A61B 1/00006; A61B 1/051

(57) ABSTRACT

A living body observation system includes a light source apparatus configured to be able to emit light in a first wavelength band, light in a second wavelength band, and light in a third wavelength band, a processor configured to perform control to emit illumination light including the lights in the first to third wavelength bands, a first image pickup device configured to have a sensitivity in each of the first and third wavelength bands, a second image pickup device configured to have a sensitivity in the second wavelength band, and a spectral optical system configured to emit the lights in the first and third wavelength bands included in reflected light from an object irradiated with the illumination light toward the first image pickup device and emit the light in the second wavelength band included in the reflected light toward the second image pickup device.

### CROSS REFERENCE TO RELATED APPLICATION

This application is a continuation application of PCT/JP2017/008107 filed on Mar. 1, 2017 and claims benefit of Japanese Application No. 2016-100593 filed in Japan on May 19, 2016, the entire contents of which are incorporated herein by this reference.

### BACKGROUND OF THE INVENTION

### Field of the Invention

The present invention relates to a living body observation system, and more particularly, to a living body observation system used to observe a blood vessel existing at a depth of a living tissue.

### Description of the Related Art

In endoscope observation in a medical field, an observation method for irradiating a living tissue with light in a red range to observe a state of a blood vessel existing at a depth of the living tissue has been conventionally proposed.

More specifically, International Publication No. 2013/145410, for example, discloses, in an endoscope apparatus, a configuration for irradiating a living tissue with narrow-band light NL1 having a wavelength in the vicinity of 600 nm, narrow-band light NL2 having a wavelength in the vicinity of 630 nm, and narrow-band light NL3 having a wavelength in the vicinity of 540 nm in a frame-sequential manner to observe a state of a blood vessel existing at a depth of the living tissue.

### SUMMARY OF THE INVENTION

A living body observation system according to an aspect of the present invention includes a light source apparatus configured to be able to emit light in a first wavelength band as narrow-band light which belongs to a red range in a visible range and falls between a wavelength representing a maximum value and a wavelength representing a minimum value in an absorption characteristic of hemoglobin, light in a second wavelength band as narrow-band light which belongs to a longer wavelength side than the first wavelength band and in which an absorption coefficient in the absorption characteristic of hemoglobin is lower than an absorption coefficient in the first wavelength band and a scattering characteristic of a living tissue is suppressed, and light in a third wavelength band as light which belongs to a shorter wavelength side than the first wavelength band, a processor configured to perform control to emit illumination light including the light in the first wavelength band, the light in the second wavelength band, and the light in the third wavelength band from the light source apparatus, a first image pickup device configured to have a sensitivity in each of the first wavelength band and the third wavelength band, a second image pickup device configured to have a sensitivity in the second wavelength band, and a spectral optical system configured to emit, when reflected light from an object irradiated with the illumination light is incident, the light in the first wavelength band and the light in the third wavelength band included in the reflected light from the object toward the first image pickup device and emit the light in the second wavelength band included in the reflected light from the object toward the second image pickup device.

### BRIEF DESCRIPTION OF THE DRAWINGS

FIG. 1 is a diagram illustrating a configuration of a principal part of a living body observation system according to an embodiment;

FIG. 2 is a diagram for describing an example of a specific configuration of the living body observation system according to the embodiment;

FIG. 3 is a diagram illustrating an example of an optical characteristic of a dichroic mirror provided in a camera unit in an endoscope according to the embodiment;

FIG. 4 is a diagram illustrating an example of a sensitivity characteristic of an image pickup device provided in the camera unit in the endoscope according to the embodiment;

FIG. 5 is a diagram illustrating an example of a sensitivity characteristic of an image pickup device provided in the camera unit in the endoscope according to the embodiment;

FIG. 6 is a diagram illustrating an example of light emitted from each of light sources provided in a light source device according to the embodiment; and

FIG. 7 is a diagram for describing an example of a specific configuration of an image processing section provided in a processor according to the embodiment.

### DETAILED DESCRIPTION OF THE PREFERRED EMBODIMENT(S)

An embodiment of the present invention will be described below with reference to the drawings.

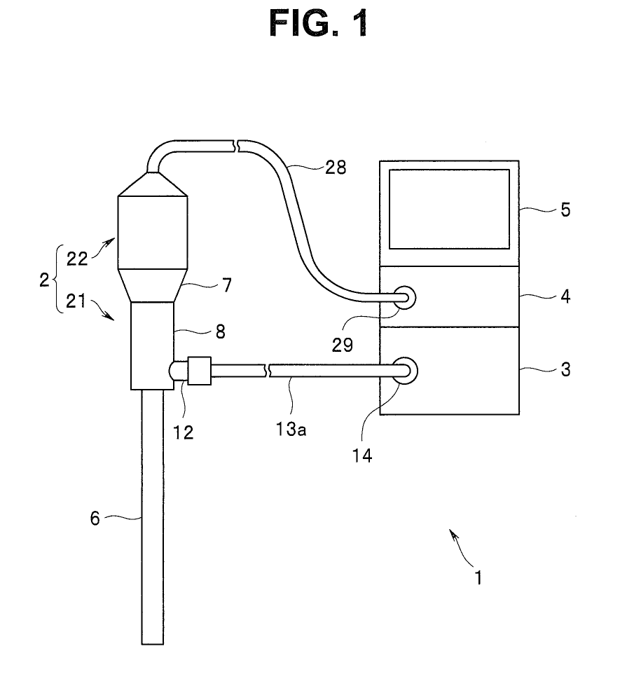

FIGS. 1 to 7 each relate to an embodiment of the present invention.

A living body observation system 1 as an endoscope apparatus includes an endoscope 2 configured to be inserted into a subject while picking up an image of an object such as a living tissue within the subject to output an image signal, a light source device 3 configured to supply light irradiated onto the object to the endoscope 2, a processor 4 configured to generate and output an observation image based on the image signal outputted from the endoscope 2, and a display device 5 configured to display the observation image outputted from the processor 4 on a screen, as illustrated in FIG. 1. FIG. 1 is a diagram illustrating a configuration of a principal part of the living body observation system according to the embodiment.

The endoscope 2 includes an optical viewing tube 21 including an elongated insertion section 6 and a camera unit 22 removably mountable on an eyepiece section 7 in the optical viewing tube 21.

The optical viewing tube 21 includes the elongated insertion section 6 which is insertable into the subject, a gripping section 8 provided in a proximal end portion of the insertion section 6, and the eyepiece section 7 provided in a proximal end portion of the gripping section 8.

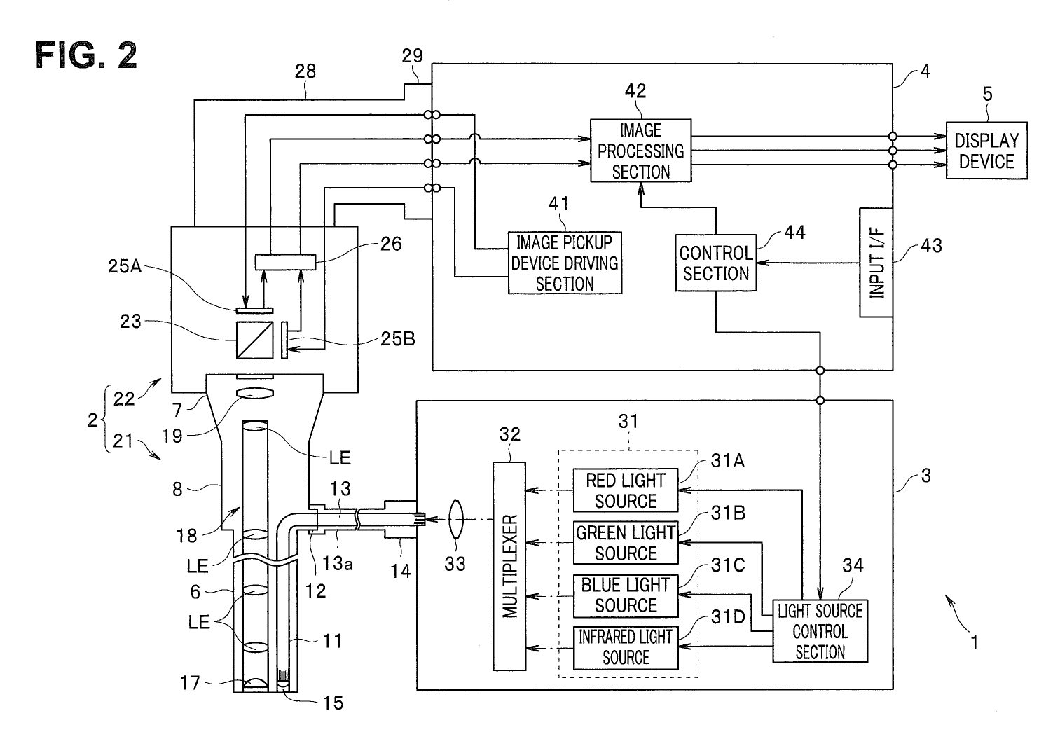

A light guide 11 for transmitting light supplied via a cable 13a is inserted, as illustrated in FIG. 2, into the insertion section 6. FIG. 2 is a diagram for describing an example of a specific configuration of the living body observation system according to the embodiment.

An emission end portion of the light guide 11 is arranged near an illumination lens 15 in a distal end portion of the insertion section 6, as illustrated in FIG. 2. An incidence end portion of the light guide 11 is also arranged in a light guide ferrule 12 provided in the gripping section 8.

A light guide 13 for transmitting light supplied from the light source device 3 is inserted, as illustrated in FIG. 2, into the cable 13a. A connection member (not illustrated) removably mountable on the light guide ferrule 12 is also provided at one of ends of the cable 13a. A light guide connector 14 removably mountable on the light source device 3 is also provided at the other end of the cable 13a.

The illumination lens 15 for emitting light transmitted from the light guide 11 to outside and an objective lens 17 for obtaining an optical image corresponding to the light to be incident from outside are provided in the distal end portion of the insertion section 6. An illumination window (not illustrated) in which the illumination lens 15 is arranged and an observation window (not illustrated) in which the objective lens 17 is arranged are provided adjacent to each other on a distal end surface of the insertion section 6.

A relay lens 18 including a plurality of lenses LE for transmitting an optical image obtained by the objective lens 17 to the eyepiece section 7 is provided, as illustrated in FIG. 2, within the insertion section 6. That is, the relay lens 18 is configured to have a function as a transmission optical system which transmits light incident from the objective lens 17.

An eyepiece lens 19 for enabling an optical image transmitted by the relay lens 18 to be observed with naked eyes is provided, as illustrated in FIG. 2, within the eyepiece section 7.

The camera unit 22 includes a dichroic mirror 23 and image pickup devices 25A and 25B.

The dichroic mirror 23 is configured to transmit light in a visible range included in light emitted via the eyepiece lens 19 toward the image pickup device 25A while reflecting light in a near-infrared range included in the emitted light toward the image pickup device 25B.

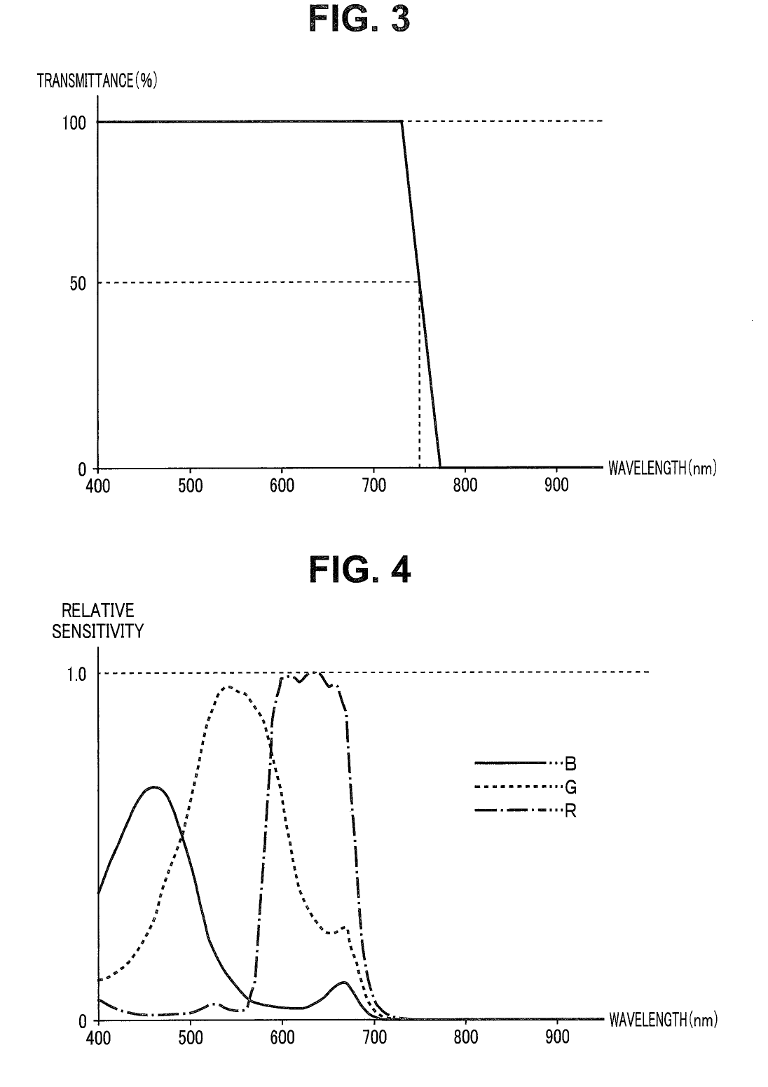

The dichroic mirror 23 is configured such that its spectral transmittance in a wavelength band belonging to the visible range becomes 100%, as shown in FIG. 3, for example. The dichroic mirror 23 is also configured such that a half-value wavelength as a wavelength at which the spectral transmittance is equal to 50% becomes 750 nm, as shown in FIG. 3, for example. FIG. 3 is a diagram illustrating an example of an optical characteristic of the dichroic mirror provided in the camera unit in the endoscope according to the embodiment.

That is, the dichroic mirror 23 has a function of a spectral optical system, and is configured such that light to be emitted via the eyepiece lens 19 is emitted by being separated into lights in two wavelength bands, i.e., light in the visible range and light in the near-infrared range.

Note that the dichroic mirror 23 may be configured such that the half-value wavelength becomes another wavelength different from 750 nm as long as the dichroic mirror 23 has the above-described function of the spectral optical system.

The image pickup device 25A includes a color CCD (charge coupled device), for example. The image pickup device 25A is also arranged at a position where the light in the visible range, which has been transmitted by the dichroic mirror 23, is receivable within the camera unit 22. The image pickup device 25A also includes a plurality of pixels for photoelectrically converting the light in the visible range which has been transmitted by the dichroic mirror 23 to pick up an image and a primary color filter provided on an image pickup surface where the plurality of pixels are arranged in a two-dimensional shape. The image pickup device 25A is also configured to be driven in response to an image pickup device driving signal outputted from the processor 4 while picking up an image of the light in the visible range which has been transmitted by the dichroic mirror 23 to generate an image pickup signal and outputting the generated image pickup signal to a signal processing circuit 26.

The image pickup device 25A is configured to have a sensitivity characteristic, as illustrated in FIG. 4, in each of wavelength bands in R (red), G (green), and B (blue). That is, the image pickup device 25A is configured to have a sensitivity in a visible range including each of the wavelength bands in R, G, and B while not or almost not having a sensitivity in a wavelength band other than the visible range. FIG. 4 is a diagram illustrating an example of a sensitivity characteristic of the image pickup device provided in the camera unit in the endoscope according to the embodiment.

The image pickup device 25B includes a monochrome CCD, for example. The image pickup device 25B is also arranged at a position where the light in the near-infrared range, which has been reflected by the dichroic mirror 23, is receivable within the camera unit 22. The image pickup device 25B also includes a plurality of pixels for photoelectrically converting the light in the near-infrared range which has been reflected by the dichroic mirror 23 to pick up an image. The image pickup device 25B is also configured to be driven in response to the image pickup device driving signal outputted from the processor 4 while picking up an image of the light in the near-infrared range which has been reflected by the dichroic mirror 23 to generate an image pickup signal and outputting the generated image pickup signal to the signal processing circuit 26.

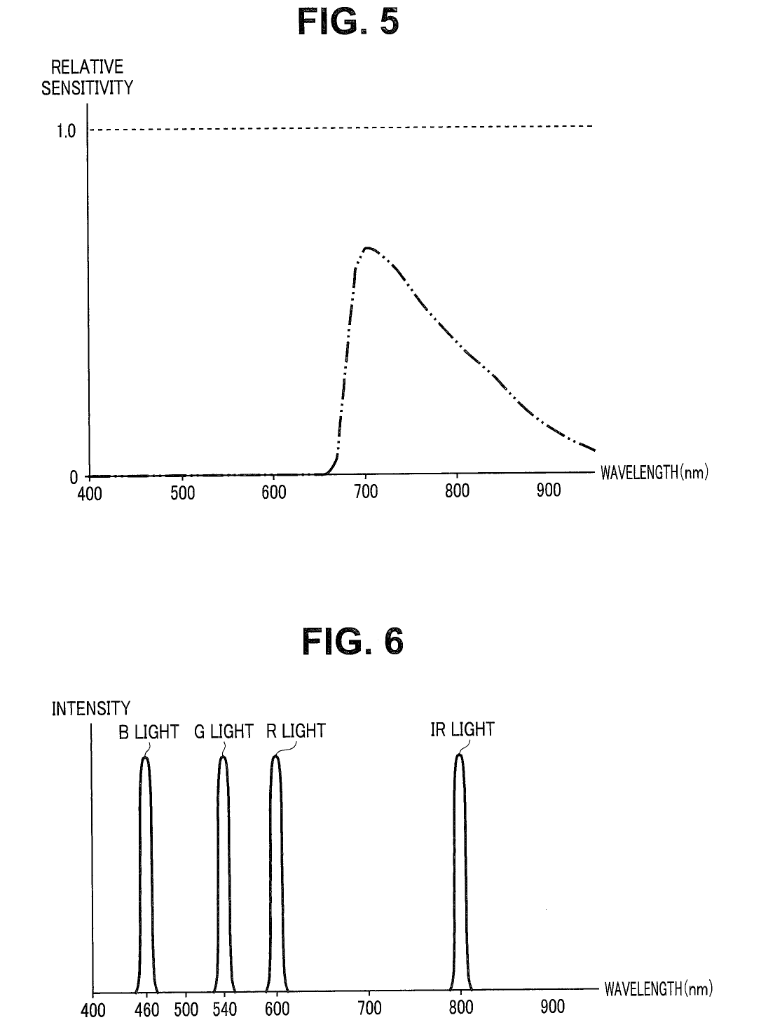

The image pickup device 25B is configured to have a sensitivity characteristic, as illustrated in FIG. 5, in the near-infrared range. More specifically, the image pickup device 25B is configured not to have or almost not to have a sensitivity in the visible range including each of the wavelength bands in R, G, and B while having a sensitivity in the near-infrared range including at least 700 nm to 900 nm, for example. FIG. 5 is a diagram illustrating an example of a sensitivity characteristic of the image pickup device provided in the camera unit in the endoscope according to the embodiment.

The signal processing circuit 26 is configured to subject the image pickup signal outputted from the image pickup device 25A to predetermined signal processing such as correlated double sampling processing and A/D (analog-to-digital) conversion processing, to generate an image signal CS including at least one of an image having a red component (hereinafter also referred to as an R image), an image having a green component (hereinafter also referred to as a G image), and an image having a blue component (hereinafter also referred to as a B image) and output the generated image signal CS to the processor 4 to which a signal cable 28 has been connected. The connector 29 is provided at an end of the signal cable 28, and the signal cable 28 is connected to the processor 4 via a connector 29. The signal processing circuit 26 is also configured to subject the image pickup signal outputted from the image pickup device 25B to predeteimined signal processing such as correlated double sampling processing and A/D conversion processing to generate an image signal IRS corresponding to an image having a near-infrared component (hereinafter also referred to as an IR image) and output the generated image signal IRS to the processor 4 to which the signal cable 28 has been connected.

Note that in the following description, for simplicity, a case where the R image and the B image included in the image signal CS have the same resolution RA and the IR image represented by the image signal IRS has a higher resolution RB than the resolution RA is taken as an example.

The light source device 3 includes a light emitting section 31, a multiplexer 32, a collecting lens 33, and a light source control section 34.

The light emitting section 31 includes a red light source 31A, a green light source 31B, a blue light source 31C, and an infrared light source 31D.

The red light source 31A includes a lamp, an LED (light emitting diode), or an LD (laser diode), for example. The red light source 31A is also configured to emit R light as narrow-band light a center wavelength and a bandwidth of which are each set to belong to a red range in the visible range and fall between a wavelength representing a maximum value and a wavelength representing a minimum value in an absorption characteristic of hemoglobin. More specifically, the red light source 31A is configured to emit R light a center wavelength and a bandwidth of which are respectively set in the vicinity of 600 nm and set to 20 nm, as illustrated in FIG. 6. FIG. 6 is a diagram illustrating an example of the light emitted from each of the light sources provided in the light source device according to the embodiment.

Note that the center wavelength of the R light is not necessarily set in the vicinity of 600 nm but may be set to a wavelength WR falling between 580 nm and 620 nm, for example. The bandwidth of the R light is not necessarily set to 20 nm but may be set to a predetermined bandwidth corresponding to the wavelength WR, for example.

The red light source 31A is configured to be switched to a lighting state or a lights-out state depending on control by the light source control section 34. The red light source 31A is also configured to generate R light having an intensity depending on the control by the light source control section 34 in the lighting state.

The green light source 31B includes a lamp, an LED, or an LD (laser diode), for example. The green light source 31B is also configured to generate G light as narrow-band light belonging to a green range. More specifically, the green light source 31B is configured to emit G light a center wavelength and a bandwidth of which are respectively set in the vicinity of 540 nm and set to 20 nm, as illustrated in FIG. 6.

Note that the center wavelength of the G light may be set to a wavelength WG belonging to the green range. The bandwidth of the G light is not necessarily set to 20 nm but may be set to a predetermined bandwidth corresponding to the wavelength WG, for example.

The green light source 31B is configured to be switched to a lighting state or a lights-out state depending on the control by the light source control section 34. The green light source 31B is also configured to generate G light having an intensity depending on the control by the light source control section 34 in the lighting state.

The blue light source 31C includes a lamp, an LED, or an LD (laser diode), for example. The blue light source 31C is also configured to generate B light as narrow-band light belonging to a blue range. More specifically, the blue light source 31C is configured to emit B light a center wavelength and a bandwidth of which are respectively set in the vicinity of 460 nm and set to 20 nm, as illustrated in FIG. 6.

Note that the center wavelength of the B light may be set in the vicinity of 470 nm, for example, as long as the center wavelength is set to a wavelength WB belonging to the blue range. The bandwidth of the B light is not necessarily set to 20 nm but may be set to a predetermined bandwidth corresponding to the wavelength WB, for example.

The blue light source 31C is configured to be switched to a lighting state or a lights-out state depending on the control by the light source control section 34. The blue light source 31C is also configured to generate B light having an intensity depending on the control by the light source control section 34 in the lighting state.

The infrared light source 31D includes a lamp, an LED, or an LD (laser diode), for example. The infrared light source 31D is also configured to emit IR light as narrow-band light a center wavelength and a bandwidth of which have been each set to belong to a near-infrared range and such that an absorption coefficient in an absorption characteristic of hemoglobin is lower than an absorption coefficient of the wavelength WR (e.g., 600 nm) and a scattering characteristic of a living tissue is suppressed. More specifically, the infrared light source 31D is configured to emit IR light a center wavelength and a bandwidth of which have been respectively set in the vicinity of 800 nm and set to 20 nm, as illustrated in FIG. 6.

Note that the above-described phrase “a scattering characteristic of a living tissue is suppressed” is intended to include a meaning that “a scattering coefficient of a living tissue decreases toward a long wavelength side”. The center wavelength of the IR light is not necessarily set in the vicinity of 800 nm but may be set to a wavelength WIR falling between 790 nm and 810 nm, for example. The bandwidth of the IR light is not necessarily set to 20 nm but may be set to a predetermined bandwidth corresponding to the wavelength WIR, for example.

The infrared light source 31D is configured to be switched to a lighting state or a lights-out state depending on the control by the light source control section 34. The infrared light source 31D is also configured to generate IR light having an intensity depending on the control by the light source control section 34 in the lighting state.

The multiplexer 32 is configured such that lights emitted from the light emitting section 31 can be multiplexed to be incident on the collecting lens 33.

The collecting lens 33 is configured such that the lights which have been incident via the multiplexer 32 are collected to be emitted to the light guide 13.

The light source control section 34 is configured to perform control for each of light sources in the light emitting section 31 based on a system control signal outputted from the processor 4.

The processor 4 includes an image pickup device driving section 41, an image processing section 42, an input IX (interface) 43, and a control section 44.

The image pickup device driving section 41 includes a driving circuit, for example. The image pickup device driving section 41 is also configured to generate and output an image pickup device driving signal for driving each of the image pickup devices 25A and 25B.

Note that the image pickup device driving section 41 may drive each of the image pickup devices 25A and 25B in response to a driving command signal from the control section 44. More specifically, the image pickup device driving section 41 may drive only the image pickup device 25A when set to a white light observation mode while driving the image pickup devices 25A and 25B when set to a deep blood vessel observation mode, for example.

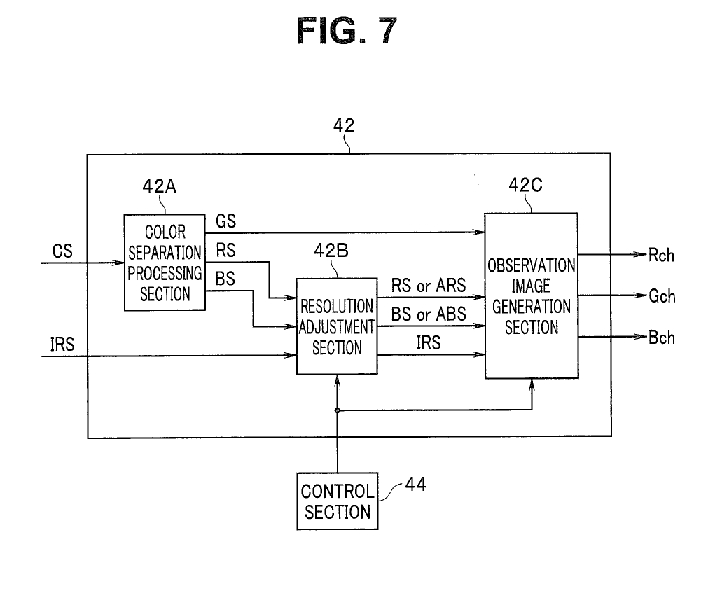

The image processing section 42 includes an image processing circuit, for example. The image processing section 42 is also configured to generate an observation image corresponding to an observation mode of the living body observation system 1 and output the generated observation image to the display device 5 based on the image signals CS and IRS outputted from the endoscope 2 and the system control signal outputted from the control section 44. The image processing section 42 also includes a color separation processing section 42A, a resolution adjustment section 42B, and an observation image generation section 42C, as illustrated in FIG. 7, for example. FIG. 7 is a diagram for describing an example of a specific configuration of the image processing section provided in the processor according to the embodiment.

The color separation processing section 42A is configured to perform color separation processing for separating the image signal CS outputted from the endoscope 2 into an R image, a G image, and a B image, for example. The color separation processing section 42A is also configured to generate an image signal RS corresponding to the R image obtained by the above-described color separation processing and output the generated image signal RS to the resolution adjustment section 42B. The color separation processing section 42A is also configured to generate an image signal BS corresponding to the B image obtained by the above-described color separation processing and output the generated image signal BS to the resolution adjustment section 42B. The color separation processing section 42A is also configured to generate an image signal GS corresponding to the G image obtained by the above-described color separation processing and output the generated image signal GS to the observation image generation section 42C.

The resolution adjustment section 42B is configured to output the image signals RS and BS outputted from the color separation processing section 42A as they are to the observation image generation section 42C when set to the white light observation mode, for example, based on the system control signal outputted from the control section 44.

The resolution adjustment section 42B is configured to perform pixel interpolation processing for increasing the resolution RA of the R image represented by the image signal RS outputted from the color separation processing section 42A until the resolution RA matches the resolution RB of the IR image represented by the image signal IRS outputted from the endoscope 2 when set to the deep blood vessel observation mode, for example, based on the system control signal outputted from the control section 44. The resolution adjustment section 42B is also configured to perform pixel interpolation processing for increasing the resolution RA of the B image represented by the image signal BS outputted from the color separation processing section 42A until the resolution RA matches the resolution RB of the IR image represented by the image signal IRS outputted from the endoscope 2 when set to the deep blood vessel observation mode, for example, based on the system control signal outputted from the control section 44.

The resolution adjustment section 42B is configured to output the image signal IRS outputted from the endoscope 2 as it is to the observation image generation section 42C when set to the deep blood vessel observation mode, for example, based on the system control signal outputted from the control section 44. The resolution adjustment section 42B is also configured to generate an image signal ARS corresponding to the R image, which has been subjected to the above-described pixel interpolating processing, when set to the deep blood vessel observation mode, for example, based on the system control signal outputted from the control section 44. The resolution adjustment section 42B is also configured to generate an image signal ABS corresponding to the B image, which has been subjected to the above-described pixel interpolating processing, and output the generated image signal ABS to the observation image generation section 42C when set to the deep blood vessel observation mode, for example, based on the system control signal outputted from the control section 44.

That is, the resolution adjustment section 42B is configured to perform processing for making the resolution of the R image represented by the image signal RS outputted from the color separation processing section 42A, the resolution of the B image represented by the image signal BS outputted from the color separation processing section 42A, and the resolution of the IR image represented by the image signal IRS outputted from the endoscope 2 match one another before the observation image is generated by the observation image generation section 42C when set to the deep blood vessel observation mode.

The observation image generation section 42C is configured to assign the R image represented by the image signal RS outputted from the resolution adjustment section 42B to an R channel corresponding to a red color of the display device 5, assign the G image represented by the image signal GS outputted from the color separation processing section 42A to a G channel corresponding to a green color of the display device 5, and assign the B image represented by the image signal BS outputted from the resolution adjustment section 42B to a B channel corresponding to a blue color of the display device 5 to generate an observation image and output the generated observation image to the display device 5 when set to the white light observation mode, for example, based on the system control signal outputted from the control section 44.

The observation image generation section 42C is configured to assign the IR image represented by the image signal IRS outputted from the resolution adjustment section 42B to the R channel corresponding to the red color of the display device 5, assign the R image represented by the image signal ARS outputted from the resolution adjustment section 42B to the G channel corresponding to the green color of the display device 5, and assign the B image represented by the image signal ABS outputted from the resolution adjustment section 42B to the B channel corresponding to the blue color of the display device 5 to generate an observation image and output the generated observation image to the display device 5 when set to the deep blood vessel observation mode, for example, based on the system control signal outputted from the control section 44.

The input UF 43 includes one or more switches and/or buttons capable of issuing an instruction, for example, in response to a user's operation. More specifically, the input I/F 43 includes an observation mode changeover switch (not illustrated) capable of issuing an instruction to set (switch) an observation mode of the living body observation system 1 to either one of the white light observation mode and the deep blood vessel observation mode in response to the user's operation, for example.

The control section 44 includes a control circuit such as a CPU (central processing unit) or an FPGA (field programmable gate array). The control section 44 is also configured to generate a system control signal for performing an operation corresponding to the observation mode of the living body observation system 1 and output the generated system control signal to the light source control section 34 and the image processing section 42 based on the instruction issued by the observation mode changeover switch in the input I/F 43.

The display device 5 includes an LCD (liquid crystal display), for example, and is configured such that the observation image or the like outputted from the processor 4 can be displayed.

An operation of the living body observation system 1 according to the embodiment will be described below.

First, a user such as an operator connects the sections in the living body observation system 1 to one another and turns on power to the sections, and then operates the input I/F 43, to issue an instruction to set an observation mode of the living body observation system 1 to a white light observation mode.

The control section 44 generates a system control signal for simultaneously emitting R light, G light, and B light from the light source device 3 and outputs the generated system control signal to the light source control section 34 when the control section 44 detects that the observation mode has been set to the white light observation mode based on an instruction issued from the input I/F 43. The control section 44 also generates a system control signal for performing an operation corresponding to the white light observation mode and outputs the generated system control signal to the resolution adjustment section 42B and the observation image generation section 42C when the control section 44 detects that the observation mode has been set to the white light observation mode based on the instruction issued from the input I/F 43.

The light source control section 34 performs control to bring the red light source 31A, the green light source 31B, and the blue light source 31C into a lighting state while performing control to bring the infrared light source 31D into a light-out state based on the system control signal outputted from the control section 44.

When the above-described operation is performed in the light source control section 34, WL light as white light including the R light, the G light, and the B light is irradiated onto an object as illumination light, and WLR light as reflected light emitted from the object in response to the irradiation with the WL light is incident from the objective lens 17 as return light. The WLR light which has been incident from the objective lens 17 is also emitted to the camera unit 22 via the relay lens 18 and the eyepiece lens 19.

The dichroic mirror 23 transmits the WLR light emitted via the eyepiece lens 19 toward the image pickup device 25A.

The image pickup device 25A picks up an image of the WLR light which has been transmitted by the dichroic mirror 23, to generate an image pickup signal and outputs the generated image pickup signal to the signal processing circuit 26.

The signal processing circuit 26 subjects the image pickup signal outputted from the image pickup device 25A to predetermined signal processing such as correlated double sampling processing and AID conversion processing, to generate an image signal CS including an R image, a G image, and a B image and output the generated image signal CS to the processor 4.

The color separation processing section 42A performs color separation processing for separating the image signal CS outputted from the endoscope 2 into the R image, the G image, and the B image. The color separation processing section 42A also outputs an image signal RS corresponding to the R image obtained by the above-described color separation processing and an image signal BS corresponding to the B image obtained by the above-described color separation processing to the resolution adjustment section 42B. The color separation processing section 42A also outputs the image signal GS corresponding to the G image obtained by the above-described color separation processing to the observation image generation section 42C.

The resolution adjustment section 42B outputs the image signals RS and BS outputted from the color separation processing section 42A as they are to the observation image generation section 42C based on the system control signal outputted from the control section 44.

The observation image generation section 42C assigns the R image represented by the image signal RS outputted from the resolution adjustment section 42B to an R channel of the display device 5, assigns the G image represented by the image signal GS outputted from the color separation processing section 42A to a G channel of the display device 5, and assign the B image represented by the image signal BS outputted from the resolution adjustment section 42B to a B channel of the display device 5 to generate an observation image and output the generated observation image to the display device 5 based on the system control signal outputted from the control section 44. According to such an operation of the observation image generation section 42C, an observation image having a substantially similar color tone to that when an object such as a living tissue is viewed with naked eyes is displayed on the display device 5.

On the other hand, the user operates the input I/F 43 with the insertion section 6 inserted into a subject and the distal end portion of the insertion section 6 arranged near a desired observation site within the subject while confirming the observation image displayed on the display device 5, to issue an instruction to set the observation mode of the living body observation system 1 to a deep blood vessel observation mode.

The control section 44 generates a system control signal for simultaneously emitting the R light, the B light, and the IR light from the light source device 3 and outputs the generated system control signal to the light source control section 34 when the control section 44 detects that the observation mode has been set to the deep blood vessel observation mode based on the instruction issued from the input I/F 43. The control section 44 also generates a system control signal for performing an operation corresponding to the deep blood vessel observation mode and outputs the generated system control signal to the resolution adjustment section 42B and the observation image generation section 42C when the control section 44 detects that the observation mode has been set to the deep blood vessel observation mode based on the instruction issued from the input I/F 43.

The light source control section 34 performs control to bring the red light source 31A, the blue light source 31C, and the infrared light source 31D into a lighting state while performing control to bring the green light source 31B into a light-out state based on the system control signal outputted from the control section 44.

When the above-described operation is performed in the light source control section 34, SL light as illumination light including the R light, the B light, and the IR light is irradiated onto the object, and SLR light as reflected light emitted from the object in response to the irradiation of the SL light is incident from the objective lens 17 as return light. The SLR light which has been incident from the objective lens 17 is also emitted to the camera unit 22 via the relay lens 18 and the eyepiece lens 19.

The dichroic mirror 23 transmits the R light and the B light included in the SLR light emitted via the eyepiece lens 19 toward the image pickup device 25A while reflecting the IR light included in the SLR light toward the image pickup device 25B.

The image pickup device 25A picks up an image of the R light and the B light which have been transmitted by the dichroic mirror 23 to generate an image pickup signal and outputs the generated image pickup signal to the signal processing circuit 26.

The image pickup device 25B picks up an image of the IR light which has been reflected by the dichroic mirror 23 to generate an image pickup signal and outputs the generated image pickup signal to the signal processing circuit 26.

The signal processing circuit 26 subjects the image pickup signal outputted from the image pickup device 25A to predetermined signal processing such as correlated double sampling processing and A/D conversion processing, to generate an image signal CS including the R image and the B image and output the generated image signal CS to the processor 4. The signal processing circuit 26 also subjects the image pickup signal outputted from the image pickup device 25B to predeteimined signal processing such as correlated double sampling processing and A/D conversion processing, to generate an image signal IRS corresponding to an IR image and output the generated image signal IRS to the processor 4.

The color separation processing section 42A performs color separation processing for separating the image signal CS outputted from the endoscope 2 into the R image and the B image. The color separation processing section 42A also outputs the image signal RS corresponding to the R image obtained by the above-described color separation processing and the image signal BS corresponding to the B image obtained by the above-described color separation processing to the resolution adjustment section 42B.

The resolution adjustment section 42B outputs the image signal IRS outputted from the endoscope 2 as it is to the observation image generation section 42C based on the system control signal outputted from the control section 44. The resolution adjustment section 42B also performs pixel interpolation processing for increasing a resolution RA of the R image represented by the image signal RS outputted from the color separation processing section 42A to a resolution RB to generate an image signal ARS corresponding to the R image which has been subjected to the pixel interpolation processing and output the generated image signal ARS to the observation image generation section 42C based on the system control signal outputted from the control section 44. The resolution adjustment section 42B also performs pixel interpolation processing for increasing a resolution RA of the B image represented by the image signal BS outputted from the color separation processing section 42A to a resolution RB to generate an image signal ABS corresponding to the B image which has been subjected to the pixel interpolation processing and output the generated image signal ABS to the observation image generation section 42C based on the system control signal outputted from the control section 44.

The observation image generation section 42C assigns the IR image represented by the image signal IRS outputted from the resolution adjustment section 42B to the R channel of the display device 5, assigns the R image represented by the image signal RS outputted from the resolution adjustment section 42B to the G channel of the display device 5, and assign the B image represented by the image signal BS outputted from the resolution adjustment section 42B to the B channel of the display device 5 to generate an observation image and output the generated observation image to the display device 5 based on the system control signal outputted from the control section 44. According to such an operation of the observation image generation section 42C, an observation image in which a blood vessel having a large diameter existing at a depth of the living tissue is emphasized in response to a contrast ratio of the R image to the IR image, for example, is displayed on the display device 5.

As described above, according to the present embodiment, the observation image in which the blood vessel having a large diameter existing at the depth of the living tissue is emphasized can be generated using the R image and the IR image obtained by simultaneously irradiating the R light and the IR light onto the living tissue in the deep blood vessel observation mode and displayed on the display device 5. Therefore, according to the present embodiment, a frame rate of the observation image displayed on the display device 5 can be more easily increased than when the R light and the IR light are irradiated in a time-divisional manner, for example. According to the present embodiment, the R image and the IR image can also be obtained by simultaneously irradiating the R light and the IR light onto the living tissue, for example. Accordingly, the R image and the IR image can be prevented from being misaligned. As a result, according to the present embodiment, image quality deterioration in an image to be displayed when a state of the blood vessel existing at the depth of the living tissue is observed can be suppressed.

According to the present embodiment, an observation image having a resolution appropriate to observe the state of the blood vessel existing at the depth of the living tissue can be generated without using a specific, less general image pickup device in which a pixel having a sensitivity in a wavelength band of the R light and a pixel having a sensitivity in a wavelength band of the IR light are arranged on the same image pickup surface, for example.

Note that according to the present embodiment, the camera unit 22 may be configured by providing such a dichroic mirror DM that its spectral transmittance in a wavelength band belonging to a visible range becomes 0 and its spectral transmittance in a wavelength band belonging to a near-infrared range becomes 100% instead of the dichroic mirror 23, arranging the image pickup device 25A at a position where light in the visible range reflected by the dichroic mirror DM is receivable, and arranging the image pickup device 25B at a position where light in the near-infrared range which has been transmitted by the dichroic mirror DM is receivable, for example.

The resolution adjustment section 42B in the present embodiment is not limited to a resolution adjustment section which performs the above-described pixel interpolation processing but may be configured to perform pixel addition processing for reducing the resolution RB of the IR image represented by the image signal IRS outputted from the endoscope 2 until the resolution RB matches the resolution RA of the R image or the B image, for example, when set to the deep blood vessel observation mode.

The configuration of each of the sections in the living body observation system 1 according to the present embodiment may be modified, as needed, so that RL light as narrow-band light having a center wavelength set in the vicinity of 630 nm and belonging to the visible range and R light as narrow-band light having a center wavelength set in the vicinity of 600 nm and belonging to the visible range are simultaneously irradiated onto the living tissue to obtain an image.

Note that the present invention is not limited to the above-described embodiment but various changes and applications may be made without departing from the scope and spirit of the invention.

What is claimed is:

1. A living body observation system comprising:
a light source apparatus configured to be able to emit light in a first wavelength band as narrow-band light which belongs to a red range in a visible range and falls between a wavelength representing a maximum value and a wavelength representing a minimum value in an absorption characteristic of hemoglobin, light in a second wavelength band as narrow-band light which belongs to a longer wavelength side than the first wavelength band and in which an absorption coefficient in the absorption characteristic of the hemoglobin is lower than an absorption coefficient in the first wavelength band and a scattering characteristic of a living tissue is suppressed, and light in a third wavelength band as light which belongs to a shorter wavelength side than the first wavelength band;
a processor configured to perform control to emit illumination light including the light in the first wavelength band, the light in the second wavelength band, and the light in the third wavelength band from the light source apparatus;
a first image pickup device configured to have a sensitivity in each of the first wavelength band and the third wavelength band;
a second image pickup device configured to have a sensitivity in the second wavelength band; and
a spectral optical system configured to emit, when reflected light from an object irradiated with the illumination light is incident, the light in the first wavelength band and the light in the third wavelength band included in the reflected light from the object toward the first image pickup device and emit the light in the second wavelength band included in the reflected light from the object toward the second image pickup device.

2. The living body observation system according to claim 1, wherein
the processor is configured to assign a first image obtained by picking up an image of the light in the first wavelength band using the first image pickup device to a first channel corresponding to a green color of a display device, assign a second image obtained by picking up an image of the light in the second wavelength band using the second image pickup device to a second channel corresponding to a red color of the display device, and assign a third image obtained by picking up an image of the light in the third wavelength band using the first image pickup device to a third channel corresponding to a blue color of the display device to generate an observation image and output the generated observation image to the display device.

3. The living body observation system according to claim 2, wherein
the processor is configured to perform processing for making a resolution of the first image, a resolution of the second image, and a resolution of the third image match one another before the observation image is generated.

4. The living body observation system according to claim 3, wherein
the processor performs pixel interpolation processing for increasing the resolution of the first image and the resolution of the third image to the resolution of the second image

5. The living body observation system according to claim 3, wherein
the processor performs pixel addition processing for reducing the resolution of the second image to the resolution of the first image or the resolution of the third image.

6. The living body observation system according to claim 1, wherein
the spectral optical system is a dichroic mirror configured to transmit the light in the first wavelength band and the light in the third wavelength band included in the reflected light from the object toward the first image pickup device and reflect the light in the second wavelength band included in the reflected light from the object toward the second image pickup device.

7. The living body observation system according to claim 1, wherein
a center wavelength of the light in the first wavelength band is set in a vicinity of 600 nm, a center wavelength of the light in the second wavelength band is set in a vicinity of 800 nm, and a center wavelength of the light in the third wavelength band is set in a vicinity of 460 nm.

8. The living body observation system according to claim 1, wherein
the light source apparatus is configured to be able to emit light in a blue range as the light in the third wavelength band and to be able to emit light in a green range as the light in the fourth wavelength band,
the processor is configured to be able to perform control to emit white light including the light in the first wavelength band, the light in the third wavelength band, and the light in the fourth wavelength band instead of the illumination light from the light source apparatus,
the first image pickup device is configured to have a sensitivity in each of the first wavelength band, the third wavelength band, and the fourth wavelength band, and
the spectral optical system is configured to emit, when the reflected light from the object irradiated with the white light is incident on the spectral system instead of the illumination light, the light in the first wavelength band, the light in the third wavelength band, and the light in the fourth wavelength band included in the reflected light from the object toward the first image pickup device.
